(19) United States
(12) Patent Application Publication
(10) Pub. No.: US 2025/0161115 A1
(43) Pub. Date: May 22, 2025

(54) SECTOR PROBE SYSTEM FOR THE DELIVERY OF LASER ENERGY

(71) Applicant: IRIDEX Corporation, Mountain View, CA (US)

(72) Inventors: Ken Arnold, Watsonville, CA (US); Jason Largent, Hayward, CA (US); Nghi Phan, San Jose, CA (US)

(73) Assignee: IRIDEX Corporation, Mountain View, CA (US)

(21) Appl. No.: 18/957,504

(22) Filed: Nov. 22, 2024

Related U.S. Application Data
(60) Provisional application No. 63/602,159, filed on Nov. 22, 2023.

(51) Int. Cl. A61F 9/008

(52) U.S. Cl. CPC A61F 9/00821

(57) ABSTRACT

A treatment probe for treating an eye of a patient includes an elongate body defining a handle and having a proximal end and a distal end, a treatment fiber configured for delivering treatment light energy to the eye from a distal end of the treatment fiber, an automated assembly for directing the treatment light energy along a treatment path to the eye from the distal end of the treatment fiber, and a tip member disposed on the distal end of the elongate body. The tip member includes one or more contact surfaces for positioning on a surface of the eye extending from the distal end of the elongate body. The treatment fiber may be housed in the elongate body.

The present application claims the benefit of priority of U.S. Provisional Appln. No. 63/602,159 filed Nov. 22, 2023, entitled, “SECTOR PROBE SYSTEM FOR THE DELIVERY OF LASER ENERGY”, which is incorporated herein by reference in its entirety for all purposes.

### BACKGROUND OF THE INVENTION

Various embodiments of the present disclosure described treatment probes for the delivery of laser energy for lowering the intraocular pressure (IOP) in human eyes afflicted with glaucoma. Various embodiments of treatment probes described herein are further directed toward laser therapy for lowering IOP in glaucomatous eyes via transconjunctival/transcleral ab-externo treatment with infrared laser energy directed to the ciliary body and pigmented ciliary epithelium, pars plicata, and/or the posterior portion of the pars plicata para plana interface.

Glaucoma is a leading cause of blindness. Glaucoma involves the loss of retinal ganglion cells in a characteristic pattern of optic neuropathy. Untreated glaucoma can lead to permanent damage of the optic nerve and resultant visual field loss, which can progress to blindness. The loss of visual field due to glaucoma often occurs gradually over a long time and may only be recognized when the loss is already quite advanced. Once lost, this damaged visual field can never be recovered.

Raised intraocular pressure (IOP) is a significant risk factor for developing glaucoma. IOP is a function of production of aqueous humor by the ciliary body of the eye and its drainage through the trabecular meshwork and all other outflow pathways including the uveoscleral pathway. Aqueous humor is a complex mixture of electrolytes, organics solutes, and other proteins that supply nutrients to the non-vascularized tissues of the anterior chamber of the eye. It flows from the ciliary bodies into the posterior chamber of the anterior segment, bounded posteriorly by the lens and the ciliary zonule and bounded anteriorly by the iris. Aqueous humor then flows through the pupil of the iris into the anterior chamber, bounded posteriorly by the iris and anteriorly by the cornea. In the conventional aqueous humor outflow path, the trabecular meshwork drains aqueous humor from the anterior chamber through the trabecular meshwork exiting into the Schlemm's canal into scleral plexuses and the general venous blood circulation. In open angle glaucoma there is reduced flow through the trabecular meshwork. In angle closure glaucoma, the iris is pushed forward against the trabeular meshwork, blocking fluid from escaping.

Uveoscleral outflow is an accessory outflow and accounts from 10-20% of total aqueous humor outflow. Enhancing uveocleral outflow is assuming a growing importance in the management of glaucoma. In uveoscleral outflow, aqueous humor enters the ciliary muscles from the anterior chamber and exits through the supraciliary space and across the anterior or posterior sclera. Uveoscleral outflow contributes significantly to total aqueous humor outflow with a reduction in intraocular pressure.

Currently, glaucoma therapies aim to reduce IOP by either limiting the production of aqueous humor or by increasing the outflow of aqueous humor. Medications such as beta-blockers, carbonic anhydrase inhibitors, etc., are used as the primary treatment to reduce the production of aqueous humor. Medications may also be used as the primary therapy to increase the outflow of the aqueous humor. Miotic and cholinergic drugs increase the trabecular outflow, while prostaglandin drugs, for example, Latanoprost and Bimatoprost, increase the uveoscleral outflow. These drugs, however, are expensive and may have undesirable side effects, which can cause compliance-dependent problems over time especially when more than one drug is prescribed.

Surgery may also be used to increase the outflow or to lower the production of aqueous humor. Laser trabeculoplasty is the application of a laser beam over areas of the trabecular meshwork to increase the outflow. Cyclocryotherapy and laser cyclophotocoagulation are surgical interventions over the ciliary processes to lower the production of aqueous humor. Although they may be effective, these destructive surgical interventions are normally used as a last resource in the management of glaucoma due to the risk of the severe complication of phthisis bulbi. Other adverse side effects of cyclodestructive surgical procedures may include temporary or permanent mydriasis (pupil dilation), ocular hypotony, inflammation of the anterior eye segment, which may be associated with an increased incidence of macula complications, and loss of best corrected visual acuity. Still other adverse side effects include transient hyphaema and exudates in the anterior chamber, uveitis, and necrotizing scleritis.

In laser transscleral cyclophotocoagulation, a continuous wave (CW) of high intensity infrared laser energy is directed toward selected portions of the pars plicata region of the ciliary body, structures under the scleral layers and the overlying conjunctiva. Selected portions of the ciliary body and related processes are permanently destroyed, thereby decreasing the overall production of aqueous humor. Laser energy may be directed through air to a patient seated at a special slit lamp. Alternatively, laser energy may be delivered through the use of fiber optic hand pieces placed in contact with the patient's eyeball. In both laser energy delivery methods, however, accurately, and repeatedly directing a laser beam a subsurface non-visible target such as the ciliary body can be challenging for a surgeon. Thus, contact hand piece probes (for example, the G-Probe available through IRIDEX Corporation of Mountain View, CA and described in U.S. Pat. No. 5,272,595, the full disclosure of which is incorporated herein by reference in its entirety) have been designed to facilitate the aiming of a laser toward the pars plicata region of the ciliary body. The G-Probe, for example, has special contours that facilitate consistent placement and aiming of the probe relative to external landmark structures of the eye (e.g., limbus), thereby guiding the laser light energy to the correct target tissue, thus likely decreasing the likelihood of adverse events and increasing the likelihood of favorable clinical responses.

While the prior systems, methods, and devices have provided advancements in the art, further improvements are desired.

### SUMMARY OF THE INVENTION

The terms “invention,” “the invention,” “this invention” and “the present invention” used in this patent are intended to refer broadly to all of the subject matter of this patent and the patent claims below. Statements containing these terms should be understood not to limit the subject matter described herein or to limit the meaning or scope of the patent claims below. Embodiments of the invention covered by this patent are defined by the claims below, not this summary. This summary is a high-level overview of various aspects of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification of this patent, any or all drawings and each claim.

According to one embodiment, a treatment probe for treating an eye of a patient includes an elongate body defining a handle and having a proximal end and a distal end, a treatment fiber configured for delivering treatment light energy to the eye from a distal end of the treatment fiber, an automated assembly for directing the treatment light energy along a treatment path to the eye from the distal end of the treatment fiber, and a tip member disposed on the distal end of the elongate body. The tip member includes one or more contact surfaces for positioning on a surface of the eye extending from the distal end of the elongate body. The treatment fiber may be housed in the elongate body. The treatment fiber may be external to the elongate body. The automated assembly may include a motorized wheel and gear mechanism. The automated assembly may include a motorized wheel mechanism. The treatment path may be an arc radius corresponding to a quadrant of the eye. The treatment probe may include an aiming beam housed in the elongate body for positioning the treatment probe relative to the eye. The tip member may be replaceable at a conclusion of a treatment session. The treatment probe may be configured to deliver a plurality of pulses of the treatment light energy to the eye to induce a therapeutic response without coagulating tissue of the eye. The treatment probe may be configured to deliver continuous wave (CW) treatment light energy to the eye to induce a therapeutic response of the eye. The treatment probe may include a rechargeable battery disposed within the elongate body for powering a treatment fiber energy source coupled to the treatment fiber. The treatment probe may include a user display for displaying one or more of display parameter settings, battery information, a mode indication, and an alert indication.

According to another embodiment, a method of treating an eye of a patient includes providing a treatment probe including an elongate body having a proximal end and a distal end, a treatment fiber housed in the elongate body, an automated assembly for directing treatment light energy along a treatment path to the eye from the distal end of the treatment fiber, and a tip member disposed on an end of the elongate body. The tip member includes one or more contact surfaces for positioning on a surface of the eye extending from the distal end of the elongate body. The method includes positioning the treatment probe in a first position relative to the eye. The one or more of the contact surfaces of the tip member is positioned on a surface of the eye. The method includes maintaining the treatment probe in the first position relative to the eye and delivering treatment light energy from a distal end of the treatment fiber to treat tissue of the eye. The method may include treating a ciliary process of the eye with the treatment light energy. Positioning the treatment probe may include confirming a relative position of the treatment probe to the eye using an aiming beam disposed within the elongate body of the treatment probe. Delivering the treatment light energy from the distal end of the treatment fiber may include delivering a plurality of pulses of the light energy so as to induce a therapeutic response without coagulating the tissue of the eye. Delivering the treatment light energy from the distal end of the treatment fiber may include delivering continuous wave (CW) treatment light energy to the eye to induce a therapeutic response of the eye. The treatment path may include an arc radius corresponding to a quadrant of the eye. The method may include positioning the treatment probe in a second position relative to the eye. The one or more of the contact surfaces of the tip member may be positioned at a different location on the surface of the eye. The method may include maintaining the treatment probe in the second position relative to the eye and delivering treatment light energy from the distal end of the treatment fiber to treat tissue of the eye.

According to another embodiment, a treatment probe for treating an eye of a patient includes an elongate body defining a handle and having a proximal end and a distal end, one or more treatment fibers housed in the elongate body and configured for delivering treatment light energy to the eye from a distal end of each of the one or more treatment fibers, an automated assembly for directing the treatment light energy along a treatment path to the eye from the distal end of each of the one or more treatment fibers, and a tip member disposed on the distal end of the elongate body. The tip member includes one or more contact surfaces for positioning on a surface of the eye extending from the distal end of the elongate body. The one or more treatment fibers may be arranged linearly along the treatment path. The automated assembly may direct the treatment light energy individually through each of the one or more treatment fibers along the treatment path.

According to yet another embodiment, a treatment probe for treating an eye of a patient includes an elongate body defining a handle and having a proximal end and a distal end, a treatment fiber housed in the elongate body and configured for delivering treatment light energy to the eye from a distal end of the treatment fiber, a rotating assembly for directing the treatment light energy along a treatment path to the eye from the distal end of the treatment fiber and through a prism of the rotating assembly, and a tip member disposed on the distal end of the elongate body. The tip member includes one or more contact surfaces for positioning on a surface of the eye extending from the distal end of the elongate body. The treatment probe may include a linear stage coupled to the rotating assembly for translating the rotating assembly radially outward from or radially inward toward a longitudinal axis of the treatment probe for adjusting the treatment path of the treatment probe. The treatment path may be an arc radius corresponding to a quadrant of the eye. Translating the rotating assembly radially outward from the longitudinal axis of the treatment probe increases the arc radius of the treatment path. Translating the rotating assembly radially inward toward the longitudinal axis of the treatment probe decreases the arc radius of the treatment path. The treatment probe may include an aiming beam housed in the elongate body for positioning the treatment probe relative to the eye. The tip member may be replaceable at a conclusion of a treatment session. The treatment probe may be configured to deliver a plurality of pulses of the treatment light energy to the eye to induce a therapeutic response without coagulating tissue of the eye. The treatment probe may be configured to deliver continuous wave (CW) treatment light energy to the eye to induce a therapeutic response of the eye. The prism of the rotating assembly may bend the treatment light energy away from an axis of the treatment fiber and toward the eye. The treatment probe may include a rechargeable battery disposed within the elongate body for powering a treatment fiber energy source coupled to the treatment fiber. The one or more contact surfaces may be cylindrically formed to form an opening at a distal end of the tip member for directing the treatment light energy to the eye. The one or more contact surfaces may form a tapered cylinder having an opening at a distal end of the tip member for directing the treatment light energy to the eye. The tapered cylinder may be partially cut on a bias relative to an axis of the treatment probe. The treatment probe may include a user display for displaying one or more of display parameter settings, battery information, a mode indication, and an alert indication. The mode indication may display an indication that the treatment probe is in a standby mode or a treatment mode. The alert indication may display an alert indicative of a treatment start, a treatment end, or an error. The treatment probe may include an accelerometer configured to detect movement of the treatment probe. The treatment light energy may be a laser beam. The treatment light energy may be a light emitting diode (LED).

According to yet another embodiment, a method of treating an eye of a patient includes providing a treatment probe including an elongate body having a proximal end and a distal end, a treatment fiber housed in the elongate body, a rotating assembly for directing treatment light energy to the eye from the distal end of the treatment fiber and through a prism of the rotating assembly, and a tip member disposed on an end of the elongate body. The tip member includes one or more contact surfaces for positioning on a surface of the eye extending from the distal end of the elongate body. The method includes positioning the treatment probe in a first position relative to the eye. The one or more of the contact surfaces of the tip member is positioned on a surface of the eye. The method includes maintaining the treatment probe in the first position relative to the eye and delivering treatment light energy from a distal end of the treatment fiber to treat tissue of the eye. The method may also include treating a ciliary process of the eye with the treatment light energy. Positioning the treatment probe may include confirming a relative position of the treatment probe to the eye using an aiming beam disposed within the elongate body of the treatment probe. Delivering the treatment light energy may include delivering the treatment light energy through the prism of the rotating assembly to bend the treatment light energy away from an axis of the treatment fiber and toward the eye. Delivering the treatment light energy from the distal end of the treatment fiber may include delivering a plurality of pulses of the light energy so as to induce a therapeutic response without coagulating the tissue of the eye. Delivering the treatment light energy from the distal end of the treatment fiber may include delivering continuous wave (CW) treatment light energy to the eye to induce a therapeutic response of the eye. Delivering treatment light energy from the distal end of the treatment fiber may include rotating the rotating assembly along a treatment path. The treatment path may be an arc radius corresponding to a quadrant of the eye. The method may include, using a linear stage coupled to the rotating assembly, translating the rotating assembly radially outward from or radially inward toward a longitudinal axis of the treatment probe for adjusting the arc radius of the treatment path. Translating the rotating assembly radially outward from the longitudinal axis of the treatment probe increases the arc radius of the treatment path. Translating the rotating assembly radially inward toward the longitudinal axis of the treatment probe decreases the arc radius of the treatment path. Positioning the contact surface on the surface of the eye may include positioning a reference structure of the one or more contact surfaces in alignment with a reference feature of the eye. The method may include positioning the treatment probe in a second position relative to the eye. The one or more of the contact surfaces of the tip member may be positioned at a different location on the surface of the eye. The method may include maintaining the treatment probe in the second position relative to the eye and delivering treatment light energy from the distal end of the treatment fiber to treat tissue of the eye.

According to another embodiment, a treatment probe for treating an eye of a patient includes an elongate body defining a handle having a proximal end and a distal end and a plurality of treatment fibers housed in the elongate body and arranged in an arc radius. The plurality of treatment fibers are configured for delivering treatment light energy to the eye from a distal end of the treatment fiber. The treatment probe includes a tip member disposed on an end of the elongate body including one or more contact surfaces for positioning on a surface of the eye extending from the distal end of the elongate body.

According to one embodiment, a system for treating an eye of a patient includes a treatment probe including an elongate body having a proximal end and a distal end, a treatment fiber housed in the elongate body and configured for delivering treatment light energy to the eye from a distal end of the treatment fiber, a rotating assembly for directing the treatment light energy to the eye from the distal end of the treatment fiber and through a prism of the rotating assembly, and a tip member disposed on an end of the elongate body. The tip member includes one or more contact surfaces for positioning on a surface of the eye extending from the distal end of the elongate body. The system includes a base station in electrical communication with the treatment probe. The base station may include a cavity shaped for and configured to hold the treatment probe when not in use. The base station may be configured to charge the treatment probe when the treatment probe is placed in the cavity. The base station may be configured to load treatment parameters to a controller disposed within the treatment probe. The base station may further include a user display. The user display may display one or more of display parameter settings, battery information, a mode indication, and an alert indication. The mode indication may display an indication that the treatment probe is in a standby mode or a treatment mode. The alert indication may display an alert indicative of a treatment start, a treatment end, or an error. The system may include a footswitch in electrical communication with the treatment probe and/or the base station. The footswitch may be configured to activate the treatment probe. The electrical communication may include Bluetooth and/or wireless internet.

According to another embodiment, a method of treating an eye of a patient includes providing a treatment probe including an elongate body having a proximal end and a distal end, a treatment fiber housed in the elongate body, a rotating assembly for directing treatment light energy to the eye from the distal end of the treatment fiber and through a prism of the rotating assembly, and a tip member disposed on an end of the elongate body. The tip member includes one or more contact surfaces for positioning on a surface of the eye extending from the distal end of the elongate body. The method includes positioning the treatment probe relative to the eye. The one or more of the contact surfaces of the tip member is positioned on a surface of the eye. The method includes maintaining the treatment probe relative to the eye and delivering treatment light energy from a distal end of the treatment fiber to a first segment of a treatment path to treat tissue of the eye. While maintaining the treatment probe relative to the eye, the method includes delivering treatment light energy from the distal end of the treatment fiber to a second segment of a treatment path to treat the tissue of the eye.

According to yet another embodiment, a method of treating an eye of a patient includes providing a treatment probe including an elongate body having a proximal end and a distal end, a treatment fiber housed in the elongate body, a rotating assembly for directing treatment light energy to the eye from the distal end of the treatment fiber and through a prism of the rotating assembly, and a tip member disposed on an end of the elongate body. The tip member includes one or more contact surfaces for positioning on a surface of the eye extending from the distal end of the elongate body. The method includes positioning the treatment probe in a first position relative to the eye. The one or more of the contact surfaces of the tip member is positioned on a surface of the eye. The method includes maintaining the treatment probe in the first position relative to the eye and delivering treatment light energy from a distal end of the treatment fiber to a first segment of a treatment path to treat tissue of the eye. The method includes repositioning the treatment probe in a second position relative to the eye. The one or more of the contact surfaces of the tip member is positioned on the surface of the eye. The method includes maintaining the treatment probe in the second position relative to the eye and delivering treatment light energy from the distal end of the treatment fiber to a second segment of a treatment path to treat the tissue of the eye.

According to another embodiment, a method of treating an eye of a patient includes providing a treatment probe including an elongate body having a proximal end and a distal end, a treatment fiber housed in the elongate body, a rotating assembly for directing treatment light energy to the eye from the distal end of the treatment fiber and through a prism of the rotating assembly, and a tip member disposed on an end of the elongate body. The tip member includes one or more contact surfaces for positioning on a surface of the eye extending from the distal end of the elongate body. The method includes positioning the treatment probe relative to the eye. The one or more of the contact surfaces of the tip member is positioned on a surface of the eye. The method includes maintaining the treatment probe relative to the eye and delivering one or more treatments of treatment light energy from a distal end of the treatment fiber to treat tissue of the eye.

According to yet another embodiment, a treatment probe for treating an eye of a patient includes an elongate body defining a handle and having a proximal end and a distal end, a treatment beam configured for delivering treatment light energy to the eye from a distal end of the treatment beam, an automated assembly for directing the treatment light energy along a treatment path to the eye from the distal end of the treatment beam, and a tip member disposed on the distal end of the elongate body. The tip member includes one or more contact surfaces for positioning on a surface of the eye extending from the distal end of the elongate body.

### BRIEF DESCRIPTION OF THE DRAWINGS

A further understanding of the nature and advantages of various embodiments may be realized by reference to the following figures. In the appended figures, similar components or features may have the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

FIG. 1 illustrates general anatomy of an eye of a patient.

FIG. 2 illustrates a simplified schematic diagram of a laser treatment system, in accordance with one embodiment of the present invention.

FIG. 3A illustrates an exterior of an exemplary treatment probe, in accordance with one embodiment of the present invention.

FIG. 3B illustrates an interior of an exemplary treatment probe, in accordance with one embodiment of the present invention.

FIG. 3C illustrates a rotating assembly of an exemplary treatment probe, in accordance with one embodiment of the present invention.

FIG. 4A illustrates a front perspective view of interior components of an exemplary treatment probe, in accordance with one embodiment of the present invention.

FIG. 4B illustrates a rear perspective view of interior components of an exemplary treatment probe, in accordance with one embodiment of the present invention.

FIG. 4C illustrates a side view of interior components of an exemplary treatment probe, in accordance with one embodiment of the present invention.

FIG. 4D illustrates an alternative interior of an exemplary treatment probe, in accordance with one embodiment of the present invention.

FIG. 5 illustrates a user display of an exemplary treatment probe, in accordance with one embodiment of the present invention.

FIG. 6A illustrates a treatment procedure using an exemplary treatment probe, in accordance with one embodiment of the present invention.

FIG. 6B illustrates a treatment procedure using an exemplary treatment probe, in accordance with one embodiment of the present invention.

FIG. 6C treatment probe of FIGS. 3A-3C positioned against an eye, in accordance with one embodiment of the present invention.

FIG. 7 illustrates an exemplary treatment probe system, in accordance with one embodiment of the present invention.

FIG. 8 illustrates a method of treating an eye of a patient, in accordance with one embodiment of the present invention.

FIGS. 9-14 illustrate embodiments of a treatment probe, in accordance with various embodiments of the present invention.

### DETAILED DESCRIPTION OF THE INVENTION

The ensuing description provides exemplary embodiments only, and is not intended to limit the scope, applicability, or configuration of the disclosure. Rather, the ensuing description of the exemplary embodiments will provide those skilled in the art with an enabling description for implementing one or more exemplary embodiments, it being understood that various changes may be made in the function and arrangement of elements without departing from the spirit and scope of the invention as set forth in the appended claims.

Various embodiments of the present disclosure provide a treatment probe and a treatment probe system having automated sweeping capabilities. In present procedures, operators use manual sweeping to apply treatment light energy along a treatment path. Operators are subject to human error including uncontrollable reflexes and/or inconsistent speed, spacing, and paths. Accordingly, the present disclosure provides systems, methods, and devices for automated sweeping where the treatment probe is held in one position by the operator and the treatment probe performs the sweeping with proper and consistent speed such that the treatment light energy is delivered in a consistent manner.

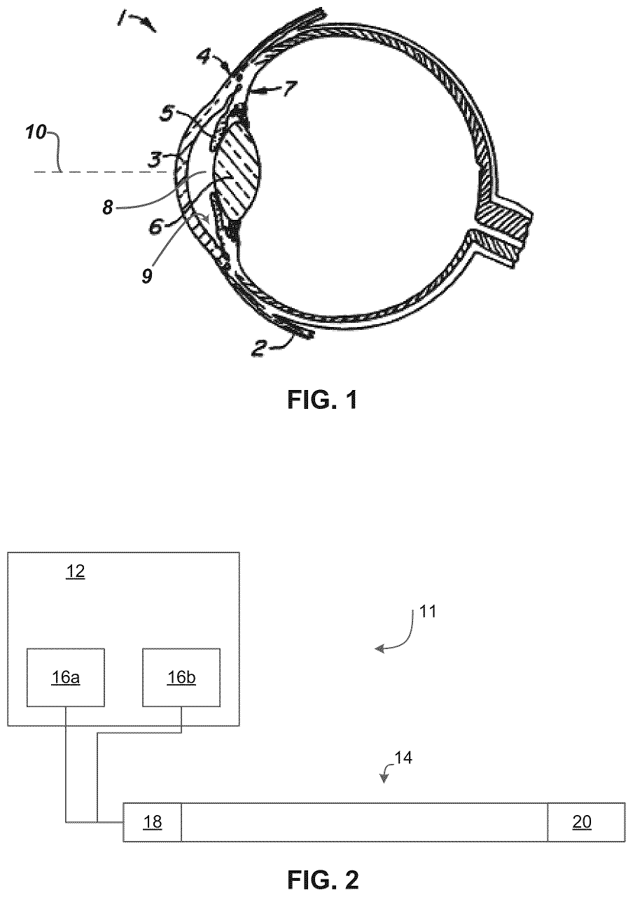

FIG. 1 illustrates the anatomy of an eye 1 with relevant parts labeled to provide anatomical references. The sclera 2 is a tough sheath around the eye which meets the cornea 3 at a circular junction called the limbus 4. Behind the cornea 3 lies the iris 5, the lens 6 and the ciliary body and related processes 7. The anterior chamber is the fluid-filled compartment within the eye 1 just in front of the pupil 8. Viewed in profile, the anterior chamber is bounded by the domed cornea 3 in front and by the colored iris 5 behind. Where the cornea 3 and the iris 5 converge they form an angle 9 referred to herein as the angle of the anterior chamber. Further eye 1 may have a visual/optical axis 10.

Embodiments described herein provide systems, methods, and devices for achieving trans-illumination of the ciliary process 7 during the application of treatment lasers (e.g., infrared laser power) to the ciliary process 7. While prior treatment methods and devices were able to generally estimate the location of the ciliary process 7 based solely on an offset distance from a patient's limbus 4, it has been found that the distance from the limbus 4 to the ciliary process 7 can vary significantly from patient to patient. Thus, illumination of the ciliary process 7 during the application of a treatment laser may provide a visual indication to an operator as to the exact location of the ciliary process 7. Thus, operators may account for the anatomical variations between patients and facilitate more accurate treatments of the ciliary process 7.

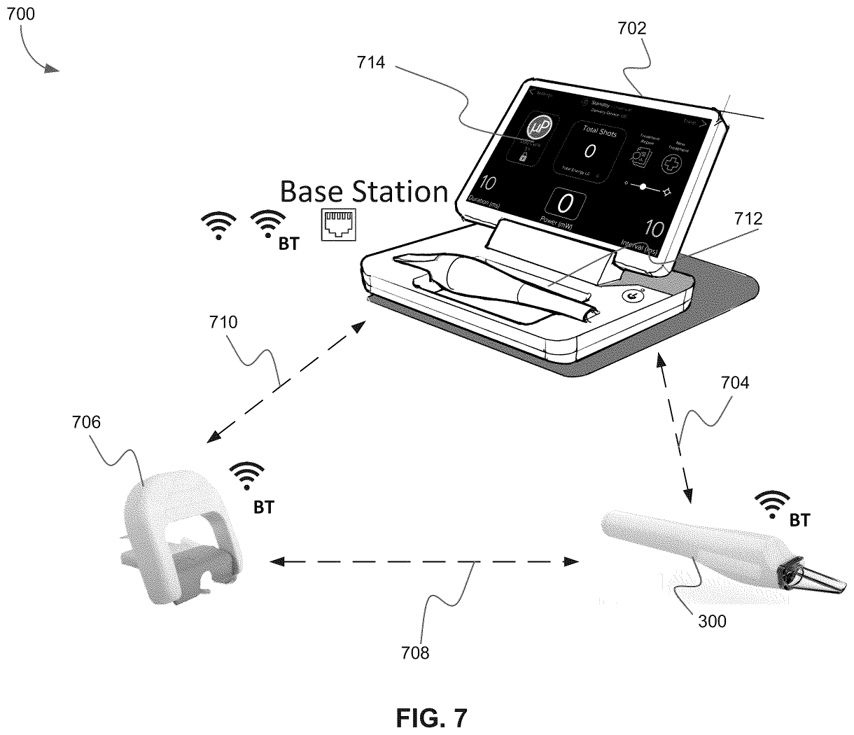

FIG. 2 illustrates a simplified schematic diagram of an exemplary system 11 for treating an eye of a patient for glaucoma according to some embodiments of the invention. Exemplary system 11 may comprise a base station 12 coupled with a treatment probe 14. Base station 12 may be configured to generate a treatment laser for treating the eye 1. The base station 12 may, for example, generate an 810 nm infrared treatment laser. Additionally, in some embodiments, base station 12 may be configured to generate illumination light (e.g., white light) for illuminating various parts of the eye 1. In some embodiments, base station 12 includes one or more outlets 16a, 16b for outputting the treatment laser and/or illumination light generated by base station 12 to a coupled treatment probe 14. The one or more outlets 16a, 16b may be configured to couple with the treatment probe 14 by receiving a proximal connection end 18 of the treatment probe 14. For example, in some embodiments, outlet 16a may output a treatment laser to a coupled treatment probe 14 for delivery to an eye 1 and outlet 16b may output the illumination light to a coupled treatment probe 14 for illumination of the ciliary process of the eye 1. In various embodiments, such as those shown and described herein, the system is wireless. For example, FIG. 7 details the wireless system below. Thus, a system user may selectively disconnect the treatment probe 14 from one or more of the outlets 16a, 16b. For example, when procedure does not require illumination light, the system user may easily deactivate such a feature by disconnecting the treatment probe 14 from outlet 16b. Additionally, or alternatively, the base station 12 may be configured to allow a user to selectively control a brightness of the illumination light. Once coupled to the base station 12, treatment probe 14 may deliver the treatment laser and/or illumination light from the base station 12 to a distal treatment end 20 of the treatment probe 14 via optical waveguides, fiber optics, light conduits, light guides, light tubes, or the like.

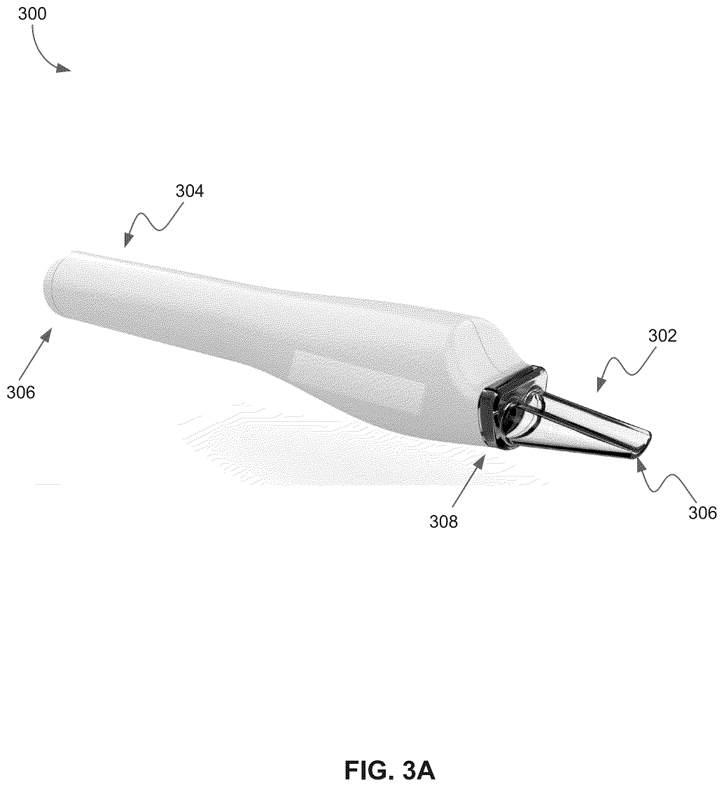

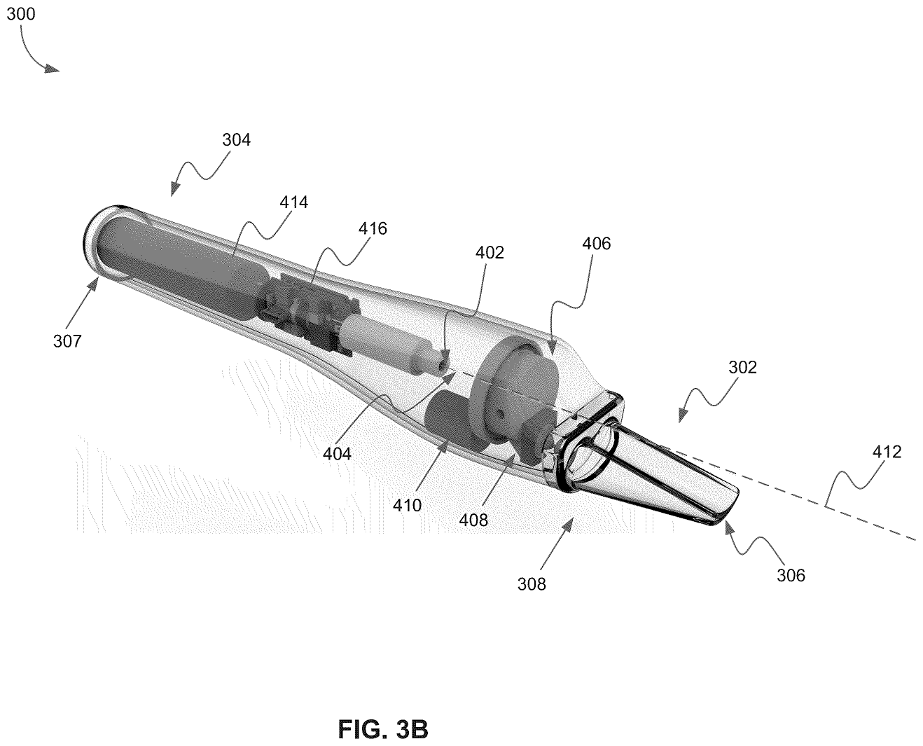

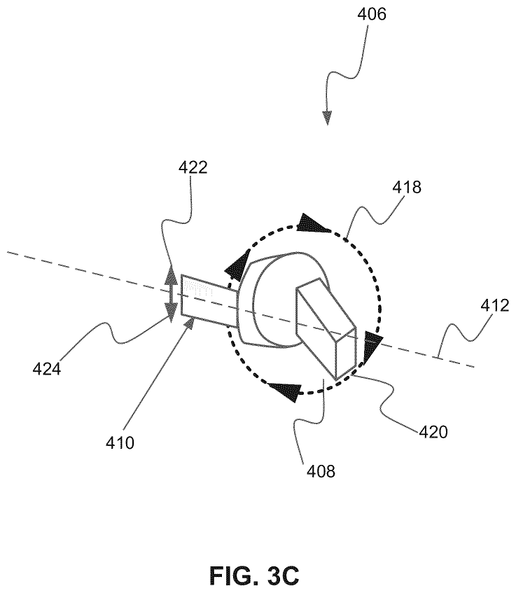

FIG. 3A illustrates an exterior of an exemplary treatment probe for treating an eye of a patient. Treatment probe 300 includes a tip member 302 coupled with a distal end of an elongate body 304 defining a handle and having a proximal end 307 and a distal end 308. The elongate body 304 is shaped and configured to be held in the hand of an operator during treatment. The elongate body 304 may be configured for manipulation by an operator and may include one or more buttons (not shown) for controlling the delivery of the treatment laser and/or illumination light from the treatment probe 300. In various embodiments, the treatment probe 300 further includes a user display (not shown) for displaying display parameter settings, battery information, a mode indication, an alert indication, etc., or a combination thereof, to be described in further detail below. In some embodiments, the mode indication displays an indication that the treatment probe 300 is in a standby mode (e.g., a charging mode and/or neutral mode) or a treatment mode. In further embodiments, the alert indication display an alert indicative of a treatment start, a treatment end, or an error. For example, an error that triggers an alert indication may include trying to initiate a treatment without sufficient battery life to perform the procedure. In other embodiments, an error alert may include an error referring to the attachment status of the tip member 302. For example, an error alert may indicate that a tip member 302 is not attached or not properly attached and treatment cannot be performed until the error is corrected, e.g., the tip member 302 is attached or properly reattached.

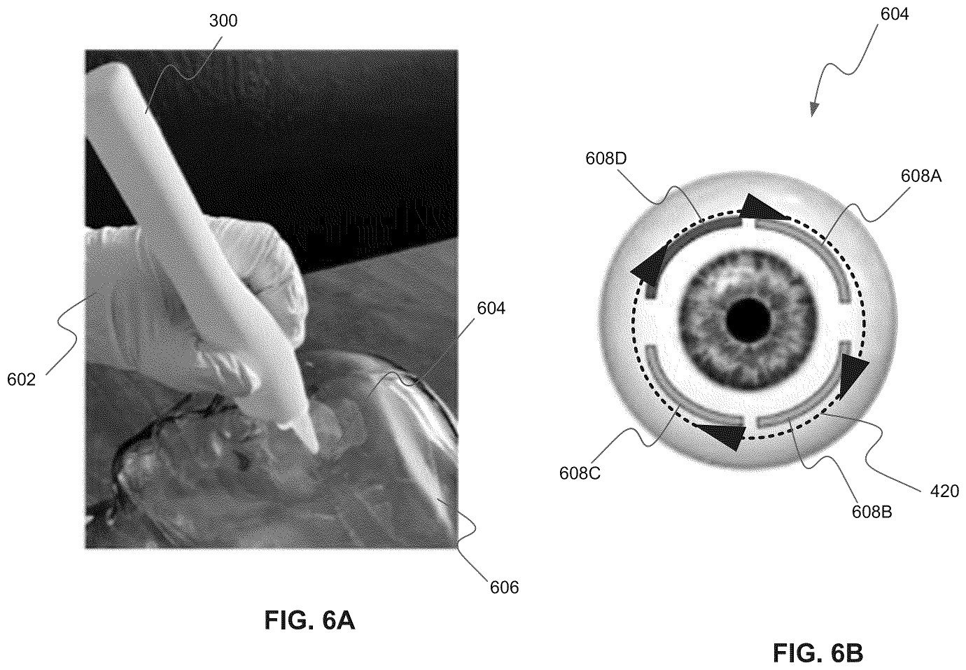

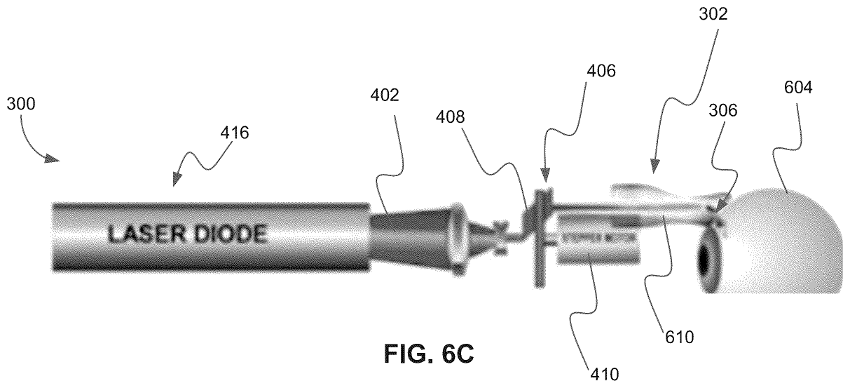

In various embodiment, the tip member 302 of the treatment probe 300 includes one or more contact surfaces 306 extending from the distal end 308 of the elongate body 304. The one or more contact surfaces 306 are used to position the treatment probe near a surface of the eye and the one or more contact surfaces 306 are positioned on the surface of the eye during treatment. According to at least some embodiments of the present disclosure, any positioning of the treatment probe 300 may include positioning and/or verifying a position of the treatment probe 300 relative to the eye using an aiming beam (not shown) housed in the treatment probe 300 and/or external to the treatment probe 300. In various embodiments, the one or more contact surfaces 306 are transparent such that an operator can view the eye and/or positioning the one or more contact surfaces 306 during positioning and/or the treatment. The one or more contact surfaces 306 may be cylindrically formed to form an opening at a distal end of the tip member 302 for directing treatment light energy to the eye. For example, the one or more contact surfaces 306 may be form a single cylinder extending from a perimeter of the tip member 302. The cylinder may have a consistent diameter throughout the length thereof or the cylinder may taper to form an opening that is relatively smaller than the distal end 308 of the elongate body 304. According to at least some embodiments, the tapered cylinder may be cut along a bias relative to an axis of a treatment fiber (to be discussed in further detail below) and/or an axis of the elongate body 304 to form a partial tapered cylinder including the one or more contact surfaces 306 such as shown in FIG. 3. In various embodiments, a shape of the one or more contact surfaces 306 corresponds to an arc radius (e.g., a segment of the treatment path). For example, the one or more contact surfaces 306 may correspond to an arc radius that is a quarter of a treatment path, described further with respect to FIG. 6B.

In at least some embodiments, the one or more contact surfaces 306 includes a plurality of contact surfaces that form legs (e.g., bunny ear-shaped legs extending from the distal end of the elongate body 304) that support the tip member 302 in position. Having a plurality of contact surfaces enables an operator to confirm the positioning of the treatment probe 300 from various angles.

In at least some embodiments, the one or more contact surfaces 306 includes a planar surface or a surface that conforms to the shape of the eye at a distal end of the tip member 302. For example, the one or more contact surfaces 306 may be a closed, transparent tip and the treatment light energy may be transmitted through a distal end of the tip member 302. In these embodiments, the closed tip may be used to push onto the tissue of the eye for removing blood during treatment.

In at least some embodiments, the tip member 302 is replaceable with a new tip member at a conclusion of a treatment session. For example, the tip member 302 may be replaced between patients, between treatment sessions, between positions, etc. In other embodiments, the tip member 302 is removable from the elongate body 304 and may be sanitized between patients, between treatment sessions, between positions, etc., in a manner that would be appreciated by one having ordinary skill in the art upon reading the present disclosure. In various embodiments, the tip member 302 may have different shapes and/or contact surfaces depending on the intended application. For example, different procedures may utilize different tip members having unique geometries as would be appreciated by one having ordinary skill in the art upon reading the present disclosure. According to at least some embodiments, a controller (not shown) disposed within the treatment probe 300 and/or within the base station (to be described in further detail below) may be able to track how many procedures and/or treatment time performed by a single tip member and a user display on the treatment probe 300 and/or the base station may alert an operator that the tip member needs to be replaced and/or discarded.

FIG. 3B illustrates an interior of an exemplary treatment probe for treating an eye of a patient. The description of treatment probe 300 with respect to FIGS. 1-3A is relevant to the description of the exemplary treatment probe of FIG. 3B and components having similar form and/or function are similarly numbered unless indicated otherwise. The interior of the treatment probe 300 includes a treatment fiber 402 housed in the elongate body 304 and configured for delivering treatment light energy to the eye from a distal end 404 of the treatment fiber 402. The treatment probe 300 described herein is configured to deliver a plurality of pulses of the treatment light energy to the eye to induce a therapeutic response without coagulating tissue of the eye. The treatment probe 300 described herein is configured to deliver continuous wave (CW) treatment light energy to the eye to induce a therapeutic response of the eye. In some embodiments, the treatment fiber 402 provides treatment light energy as a laser beam and/or a light emitting diode (LED). In other embodiments, the treatment fiber 402 may include a treatment beam from various sources.

Treatment probe 300 includes a rotating assembly 406 within the elongate body 304. Although a rotating assembly 406 is shown and described as an exemplary embodiment of the present disclosure, it is noted that the rotating assembly 406 is one embodiment of an automated assembly, to be described in further detail below, that automates the scanning associated with treatment of the eye. The rotating assembly 406 directs the treatment light energy along a treatment path to the eye from the distal end 404 of the treatment fiber 402 and through a prism 408 of the rotating assembly 406. The prism 408 of the rotating assembly 406 bends the treatment light energy away from an axis of the treatment fiber 402, which may be coincident with the longitudinal axis 412 of the elongate body 304, and toward the eye. According to various aspects of the present disclosure, the rotating assembly 406 rotates automatically during treatment along a treatment path. For example, the treatment probe 300 rotates the rotating assembly 406 according to treatment parameters loaded to the treatment probe 300 and the operator does not rotate the treatment probe 300. Rather, the operator maintains the treatment probe 300 in position as the treatment probe 300 causes the rotating assembly 406 to rotate. The speed of the rotation may be variable according to various intended applications and/or operator preferences. For example, the rotating assembly 406 rotates around an axis such that a complete rotation of the rotating assembly 406 forms a circular path. In various embodiments, the treatment probe 300 as described herein is positioned in place by an operator and the treatment fiber 402 and the rotating assembly 406 are actuated such that the treatment light energy passes through the rotating assembly 406 including the prism in a treatment path forming an arc radius. The arc radius may be the entire circumference of the range of motion of the rotating assembly 406. In at least some embodiments, a treatment path may be formed of segments such that the operator repositions the treatment probe 300 with respect to the eye. For example, and as shown and described with respect to FIG. 6B below, the treatment path may be segmented into quadrants of treatment and an arc radius may comprise one quadrant of the treatment path. Advantageously, the operator does not need to sweep the treatment probe 300 across the eye, a process that is susceptible to errors resulting from unsteady hands and/or uneven spacing and speed of the sweeping process. Accordingly, the rotating assembly 406 performs the sweeping in an accurate and consistent manner.

In at least some embodiments, the treatment probe 300 may include a plurality of treatment fibers 402 and the treatment light energy may be applied from each of the treatment fibers 402 at the same time. The treatment fibers 402 may be prearranged in an arc radius, e.g., as a segment of the treatment path, such that the treatment probe 300 does not sweep across a segment and the rotating assembly 406 rotates to treat different segments. For example, one or more treatment fibers may be housed in the elongate body and the rotating assembly (e.g., the automated assembly) directs the treatment light energy along a treatment path to the eye from the distal end of each of the one or more treatment fibers. Further, the rotating assembly may direct the treatment light energy individually through each of the one or more treatment fibers along the treatment path.

In various embodiments, the treatment fiber 402 may be coupled to the rotating assembly 406 such that the treatment fiber 402 itself is rotated during treatment.

According to various embodiments of the present disclosure, sweeping and delivering treatment light energy may include one or more of: (1) continually rotating the rotating assembly while continually applying pulses of treatment light energy (2) continually rotating the rotating assembly while applying pulses of treatment light energy in predetermined intervals (3) discrete movements while continually applying pulses of treatment light energy and (4) discrete movements between applying pulses of treatment light energy in predetermined intervals. In some embodiments, it may be desirable to skip an area on the eye that should not be treated. Accordingly, the patterns (e.g., the treatment path and segments thereof) may be fine-tuned and complex. Furthermore, the treatment probe described herein is configured to deliver continuous wave (CW) treatment light energy to the eye to induce a therapeutic response of the eye.

The treatment probe 300 further includes a linear stage 410 coupled to the rotating assembly 406 for translating the rotating assembly 406 radially outward from or radially inward toward a longitudinal axis 412 of the treatment probe 300 for adjusting the treatment path of the treatment probe 300, e.g., by adjusting the radius of the treatment path. For example, translating the rotating assembly 406 radially outward from the longitudinal axis 412 of the treatment probe 300 increases the arc radius of the treatment path and translating the rotating assembly 406 radially inward toward the longitudinal axis 412 of the treatment probe 300 decreases the arc radius of the treatment path. Accordingly, the combination of the rotating assembly 406 and the linear stage 410 enables sweeping of the eye in two dimensions and provides closed loop position control with very high resolution.

In various embodiments, the treatment probe 300 includes a rechargeable battery 414 disposed within the elongate body 304 for providing power to the treatment fiber 402, the rotating assembly 406, and/or the linear stage 410. In particular, the rechargeable battery 414 provides power to a treatment fiber energy source 416 coupled to the treatment fiber 402. Advantageously, the treatment probe 300 does not include any cords or wiring to a base station or foot actuator (to be described in further detail below) to operate. The operator is able to safely maneuver the treatment probe 300 without being susceptible to tangles which may interrupt the treatment session. In exemplary embodiments and as shown in various FIGS., the treatment probe 300 described herein is a self-contained laser and battery system in a handpiece form factor.

According to various embodiments, the treatment probe 300 may further include various required (e.g., by regulation or otherwise) laser safety features that are fully built into the treatment probe 300 as would be appreciated by one having ordinary skill in the art upon reading the present disclosure. In at least some embodiments, the treatment probe 300 as described herein may further include position sensors (not shown) related to the sweeping motion for additional safety uniquely related to sweeping. For example, the rotating assembly 406 and/or the linear stage 410, to be described in further detail below, include position sensors. The sensors described herein are used to monitor and verify that the treatment probe 300 is working as described in further detail herein and to detect unexpected interruptions in movement. The sensors further provide position control and accuracy. A Hall effect position sensor and an optical encoder may be implemented for further monitoring and verification, as would be appreciated by one having ordinary skill in the art upon reading the present disclosure. In some embodiments, the treatment probe system described herein is a closed loop system.

FIG. 3C illustrates a rotating assembly of an exemplary treatment probe. The description of treatment probe 300 with respect to FIGS. 1-3B is relevant to the description of the rotating assembly of the exemplary treatment probe of FIG. 3C and components having similar form and/or function are similarly numbered unless indicated otherwise. Visible in this view, the rotating assembly 406 includes a prism 408 for bending the treatment light energy out of the treatment probe, such as treatment probe 300. The prism 408 may be rhomboid that directs the treatment light energy at an angle from the axis of the treatment fiber 402. For example, the rhomboid may be sized and shaped to bend the light 90° away from the longitudinal axis 412 and/or an axis of the treatment fiber 402. For example, the rhomboid prism shifts the light a predetermined distance from the center line (e.g., the arc radius). As the rotating assembly 406 rotates a first direction 418, the prism 408 rotates with the rotating assembly 406 along a treatment path 420. As further shown in FIG. 3C, a linear stage 410 is coupled to the rotating assembly 406. The linear stage 410 is coupled to the rotating assembly 406 for translating the rotating assembly 406 radially outward from or radially inward toward a longitudinal axis 412 of the treatment probe 300 for adjusting the treatment path of the treatment probe 300, e.g., by adjusting the radius of the treatment path. The linear stage 410 may travel a first direction 422 or a second direction 424 to translate the rotating assembly 406 away from the longitudinal axis 412 of the treatment probe 300 to increase the arc radius of the treatment path. The linear stage 410 may translate the rotating assembly 406 in the opposite direction toward the longitudinal axis 412 of the treatment probe 300 to decrease the arc radius of the treatment path.

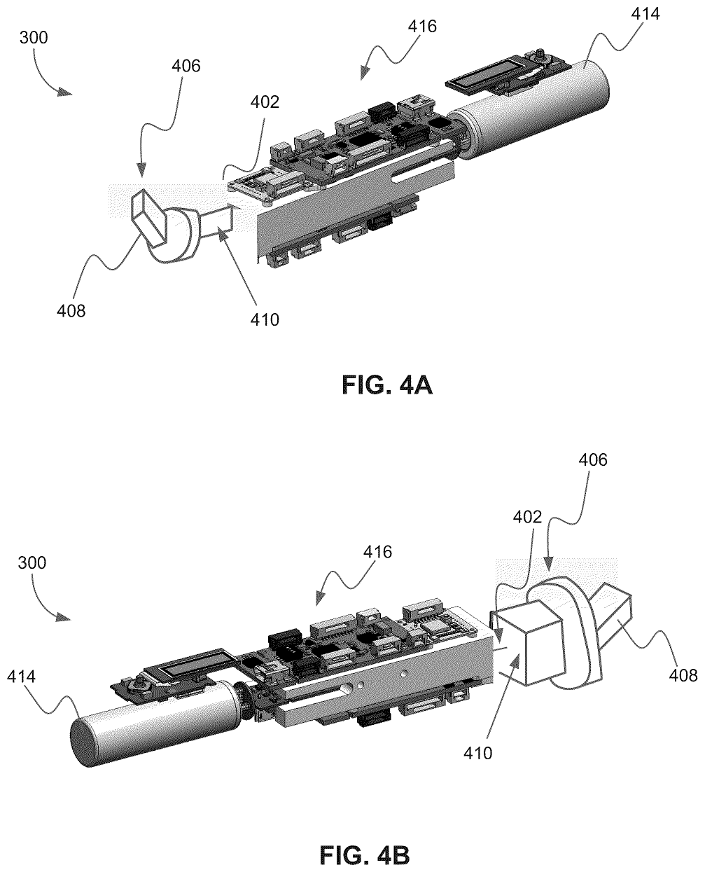

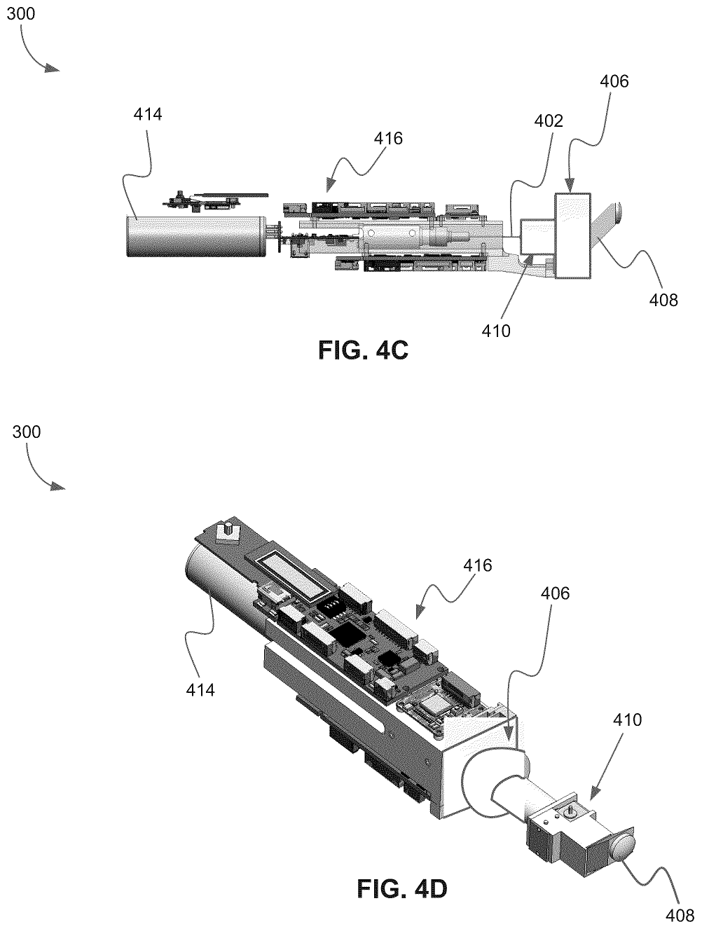

FIGS. 4A-4D illustrate additional views of the interior of the exemplary treatment probe described in detail above. For example, FIG. 4A illustrates a front perspective view of interior components of an exemplary treatment probe, FIG. 4B illustrates a rear perspective view of interior components of an exemplary treatment probe, FIG. 4C illustrates a side perspective view of interior components of an exemplary treatment probe, and FIG. 4D illustrates an alternative interior of the treatment probe. The description of treatment probe 300 with respect to FIGS. 1-3C is relevant to the description of the interior of the exemplary treatment probe shown in FIGS. 4A-4D and components having similar form and/or function are similarly numbered unless indicated otherwise.

As shown in FIG. 4D, an alternative configuration of the various components is provided. For example, the linear stage 410 may be disposed on or otherwise coupled to a distal end of the rotating assembly and the prism 408 may be coupled to the linear stage 410. Furthermore, in this embodiment, the rechargeable battery 414 may be fit under the treatment fiber energy source 416 for a relatively compact, e.g., smaller, housing (handle) of the treatment probe 300, as desired. Accordingly, various configurations of the components described herein are contemplated by the present disclosure. The configurations shown in FIGS. 4A-4C are exemplary embodiments of the treatment probe 300 described herein.

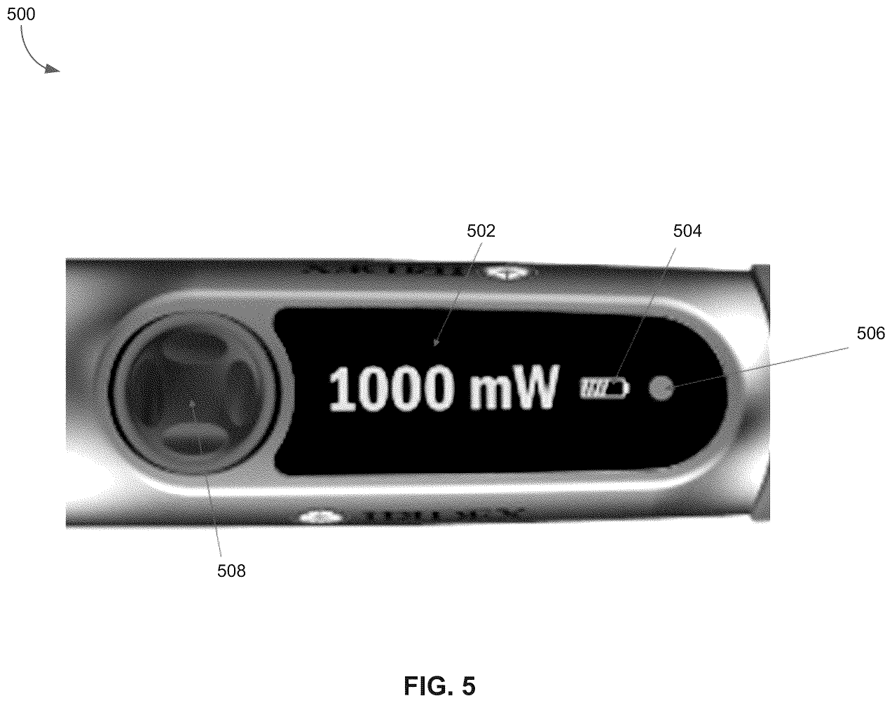

FIG. 5 illustrates a user display of an exemplary treatment probe. The description of treatment probe 300 with respect to FIGS. 1-4D is relevant to the description of the user display of the exemplary treatment probe of FIG. 5 and components having similar form and/or function are similarly numbered unless indicated otherwise. As described above, the treatment probe 300 may include a user display 500 along the elongate body 304 for displaying parameter settings 502, battery information 504, a mode indication 506, an alert indication, etc., or a combination thereof, such as shown in FIG. 5. In some embodiments, the mode indication 506 displays an indication that the treatment probe 300 is in a standby mode (e.g., a charging mode and/or neutral mode) or a treatment mode. The mode indication 506 may include text, color, shapes, or the like, or a combination thereof. For example, a treatment mode may be green, a standby mode may be blue, and an alert may be red. In further embodiments, an alert indication display an alert indicative of a treatment start, a treatment end, or an error. According to at least some embodiments, the treatment probe 300 includes an accelerometer or other movement sensing device to verify motion and/or to detect an above average “G event” (e.g., for example, if the treatment probe 300 is dropped or otherwise abused). An error alert may include an indication of a drop event. In various embodiments, the treatment probe 300 may further include one or more buttons 508 for changing a mode of the treatment probe (e.g., between a standby mode and a treatment mode and vice versa) or other functions as would be appreciated by one having ordinary skill in the art upon reading the present disclosure.

According to at least some embodiments, the treatment probe 300 may further include a speaker and/or a vibration mechanism for providing additional information to an operator such as treatment start, treatment end, turnaround points, an error, a standby/treatment mode transition, or the like.

FIG. 6A illustrates a treatment procedure using an exemplary treatment probe. The description of treatment probe 300 with respect to FIGS. 1-5 is relevant to the description of the user display of the exemplary treatment probe of FIG. 6A and components having similar form and/or function are similarly numbered unless indicated otherwise. As illustrated by FIG. 6A, an operator 602 positions the treatment probe 300 relative to an eye 604 of a patient 606 for treatment. The treatment probe 300 may be activated the operator 602 using a button on the treatment probe 300, such as the one or more buttons 508, and/or by a button or the like on a base station (to be described in further detail below), and/or by actuating a footswitch (to be described in further detail below). The operator 602 maintains the treatment probe 300 in a first position as treatment light energy is applied along a treatment path defined by the rotating assembly and/or the linear stage, such as rotating assembly 406 and linear stage 410, within the treatment probe 300. In some embodiments, the treatment path is equal to an entire rotation of the rotating assembly of the treatment probe 300 and the operator 602 maintains the treatment probe 300 relative to the eye 604 in one position for the entire treatment session. In other embodiments, the rotating assembly only rotates a portion of an entire rotation. For example, the rotating assembly may only rotate to form a quarter (e.g., a quadrant) of a circle. In these embodiments, the operator 602 maintains the treatment probe 300 relative to the eye 604 in a first position to treat a first segment and then the operator 602 readjusts the treatment probe 300 position relative to the eye 604 so that the treatment light energy may be applied to a different segment along the treatment path. According to at least some embodiments, multiple treatments may be applied to each segment of the treatment path while the treatment probe 300 is held stationary and the rotating assembly rotates within the treatment probe 300.

For example, FIG. 6B illustrates a treatment procedure using an exemplary treatment probe. The description of treatment probe 300 with respect to FIGS. 1-6A is relevant to the description of the user display of the exemplary treatment probe of FIG. 6B and components having similar form and/or function are similarly numbered unless indicated otherwise. As shown in FIG. 6B, a treatment path 420 is segmented into segments 608A-D. In the present embodiment, the treatment path 420 is segmented into four segments 608, however, any number of segments such as 2, 3, 5, etc., may be used. An operator may readjust the treatment probe for applying treatment light energy to each segment 608 along the path of the rotating assembly within the treatment probe. The segments 608 may be treated in any direction or order as would be appreciated by one having ordinary skill in the art upon reading the present disclosure.

FIG. 6C treatment probe of FIGS. 3A-3C positioned against an eye. The description of treatment probe 300 with respect to FIGS. 1-6B is relevant to the description of the user display of the exemplary treatment probe positioned against the eye as shown in FIG. 6C and components having similar form and/or function are similarly numbered unless indicated otherwise. Various components have been removed in this view for clarity. As shown in FIG. 6C, the treatment light energy 610 travels from the treatment fiber energy source 416, through the treatment fiber 402, through the prism 408 that is rotated along the treatment path by the rotating assembly 406 and directed through the tip member 302 having contact surface 306 to the eye 604 for performing the treatment.

FIG. 7 illustrates an exemplary treatment probe system. The description of treatment probe 300 with respect to FIGS. 1-6C is relevant to the description of the exemplary treatment probe system of FIG. 7 and components having similar form and/or function are similarly numbered unless indicated otherwise. In various embodiments, a treatment probe system 700 includes a treatment probe 300 and a base station 702 in electrical communication 704 with the treatment probe 300. The treatment probe system 700 may further include a footswitch 706 that is in electrical communication 708 with the treatment probe 300 and/or in electrical communication 710 with the base station 702. The electrical communication may include WiFi, Bluetooth, cables, wiring, or the like, or any combination thereof. In exemplary embodiments, no cabling, wiring, or the like are necessary between the base station 702, the treatment probe 300, and the footswitch 706 for transferring data and/or signals throughout the treatment probe system 700.

In at least some embodiments, the base station 702 is the master controller and the treatment probe 300 is responsive to accept the treatment information and enablement from the base station 702. The treatment probe 300 is used to perform the procedure and is activated by the footswitch 706 independent of the base station 702. To perform another procedure, the treatment probe 300 is docked to the base station 702 to load the treatment parameters. The base station 702 further enables the treatment probe 300 to perform the next procedure. Said another way, the treatment probe 300 functions substantially independently of the base station 702 once the treatment parameters are loaded and the treatment probe 300 is authorized to perform the treatment.

According to at least some embodiments, the base station 702 includes a cavity 712 shaped for and configured to hold the treatment probe 300 when not in use. In further embodiments, the base station 702 may include cavities for holding replacement tip members, replacement batteries, etc., In various embodiments, the base station 702 is configured to charge the treatment probe 300 (e.g., a rechargeable battery of the treatment probe 300, such as rechargeable battery 414 of the treatment probe 300). In addition to providing power to the treatment probe 300, the base station 702 may be configured to load treatment parameters to a controller disposed within the treatment probe 300. For example, an operator may schedule a treatment session in terms of parameters including the time, intensity of the treatment light energy, number of segments, etc., and the parameters may be loaded onto the treatment probe 300 when the treatment probe 300 is docked to the base station 702. The base station 702 may further include a user display 714 similar to user display 500 described with respect to FIG. 5. The user display 714 may display the same information (e.g., parameter settings 502, battery information 504, a mode indication 506, an alert indication, etc., or a combination thereof) as the user display 500 in relatively larger text. The user display 714 may include additional information that would not otherwise fit onto a user display 500 on the elongate body 304 of the treatment probe 300. In various embodiments, the user display 714 may further provide an input for an authorization of the treatment probe system 700 as would be appreciated by one having ordinary skill in the art upon reading the present disclosure.

According to various embodiments, the treatment probe system 700 includes a footswitch 706 that activates the treatment probe 300. For example, an operator may press on the footswitch with a foot and power on/off the treatment probe 300 for applying treatment light energy for the treatment. According to at least some embodiments, the treatment probe system 700 does not include the footswitch 706 and the treatment probe is activated by one or more buttons (such as one or more buttons 508 of FIG. 5).

In at least some embodiments, the treatment probe system 700 may be configured such that the treatment probe is only paired to a single base station 702 and/or footswitch 706 at a time.

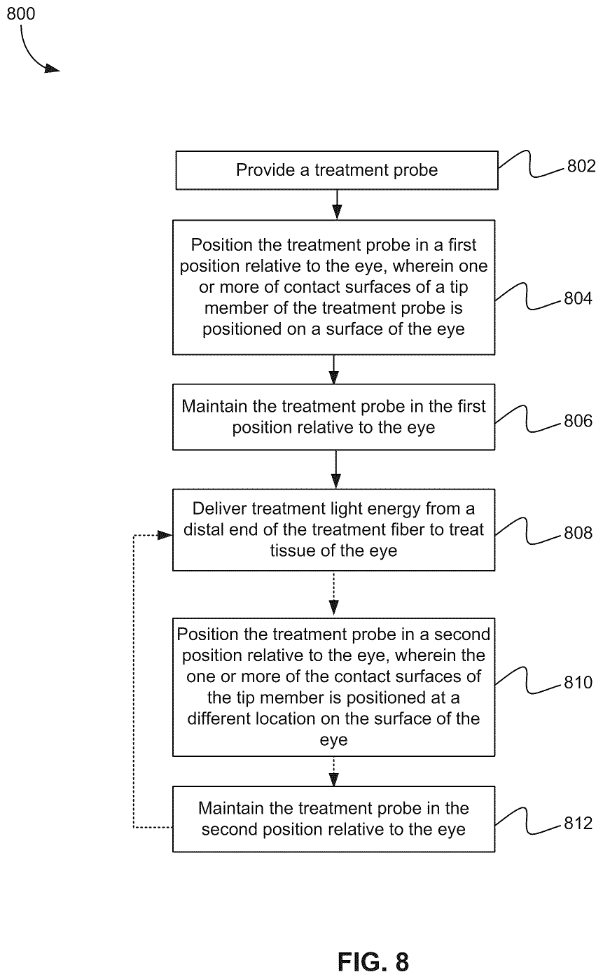

FIG. 8 illustrates a method of treating an eye of a patient. Various descriptions provided above with respect to FIGS. 1-7 are applicable to the present method 800 unless otherwise indicated. Method 800 includes step 802 including providing a treatment probe. The treatment probe may include any combination of embodiments described in detail above with respect to FIGS. 3A-7. According to various embodiments, the treatment probe includes an elongate body having a proximal end and a distal end, a treatment fiber housed in the elongate body, and a rotating assembly for directing treatment light energy to the eye from the distal end of the treatment fiber and through a prism of the rotating assembly. The treatment probe further includes a tip member disposed on an end of the elongate body. The tip member includes one or more contact surfaces for positioning on a surface of the eye. The one or more contact surfaces extend from the distal end of the elongate body.

Method 800 includes step 804 including positioning the treatment probe in a first position relative to the eye. The one or more of the contact surfaces of the tip member is positioned on a surface of the eye. According to various embodiments of the present disclosure, positioning the contact surface on the surface of the eye includes positioning a reference structure of the one or more contact surfaces in alignment with a reference feature of the eye. For example, one or more of the one or more contact surfaces may include a marking or other indicator for an operator to verify the position of the treatment probe relative to the eye. In various embodiments, the treatment probe, e.g., the one or more contact surfaces of the treatment probe, is positioned within 1 mm of the target location.

Method 800 includes step 806 including maintaining the treatment probe in the first position relative to the eye. According to various embodiments of the present disclosure, an operator maintains the treatment probe in position as the rotating assembly within the treatment probe applies the treatment in consistent manner. For example, the rotating assembly is capable of maintaining a consistent speed and energy during the sweeping process. Such automatic sweeping as described herein reduces the incidence of damage to the eye due to human error or the like. According to at least some embodiments, an operator of the treatment probe maintains the position for a time period between 2 to 5 minutes, or until the treatment of the treatment path is complete, as would be determinable by an operator in possession of the embodiments described in detail herein. The ergonomics and positioning capabilities of the treatment probe as shown and described herein enables the operator to hold the treatment probe in place for several minutes as needed for each treatment session.

Method 800 includes step 808 including delivering treatment light energy from a distal end of the treatment fiber to treat tissue of the eye. For example, step 808 includes treating a ciliary process of the eye with the treatment light energy. In various embodiments, the treatment light energy is delivered through the prism of the rotating assembly to bend the treatment light energy away from an axis of the treatment fiber and toward the eye. Delivering the treatment light energy from the distal end of the treatment fiber may include delivering a plurality of pulses of the light energy so as to induce a therapeutic response without coagulating the tissue of the eye. In other embodiments, delivery of treatment light energy from the distal end of the treatment fiber may include continuous wave (CW) treatment and/or coagulation. Step 808 further includes rotating the rotating assembly along a treatment path. In various embodiments, the rotating is performed automatically by the treatment probe as determined by the treatment parameters loaded to the treatment probe. According to various embodiments described in detail above, the treatment path may be an arc radius corresponding to a quadrant of the eye. In at least some embodiments, step 808 further includes using a linear stage coupled to the rotating assembly to translate rotating assembly radially outward from or radially inward toward a longitudinal axis of the treatment probe for adjusting the arc radius of the treatment path. As described above, translating the rotating assembly radially outward from the longitudinal axis of the treatment probe increases the arc radius of the treatment path and translating the rotating assembly radially inward toward the longitudinal axis of the treatment probe decreases the arc radius of the treatment path.

Method 800 may further include step 810 including positioning the treatment probe in a second position relative to the eye. For example, the one or more contact surfaces of the tip member may be positioned at a different location on the surface of the eye. Step 810 may include similar embodiments for the second position such as those described with respect to step 804.

Method 800 may further include step 812 including maintaining the treatment probe in the second position relative to the eye. Step 812 may include similar embodiments for the second position such as those described with respect to step 806.

As shown in FIG. 8, the method of treatment may cycle through the steps of positioning, maintaining, and delivering treatment for different positions around the eye. For example, in embodiments where the treatment path is segmented into four quadrants, the treatment probe may be repositioned four times for applying the treatment light energy to each quadrant.

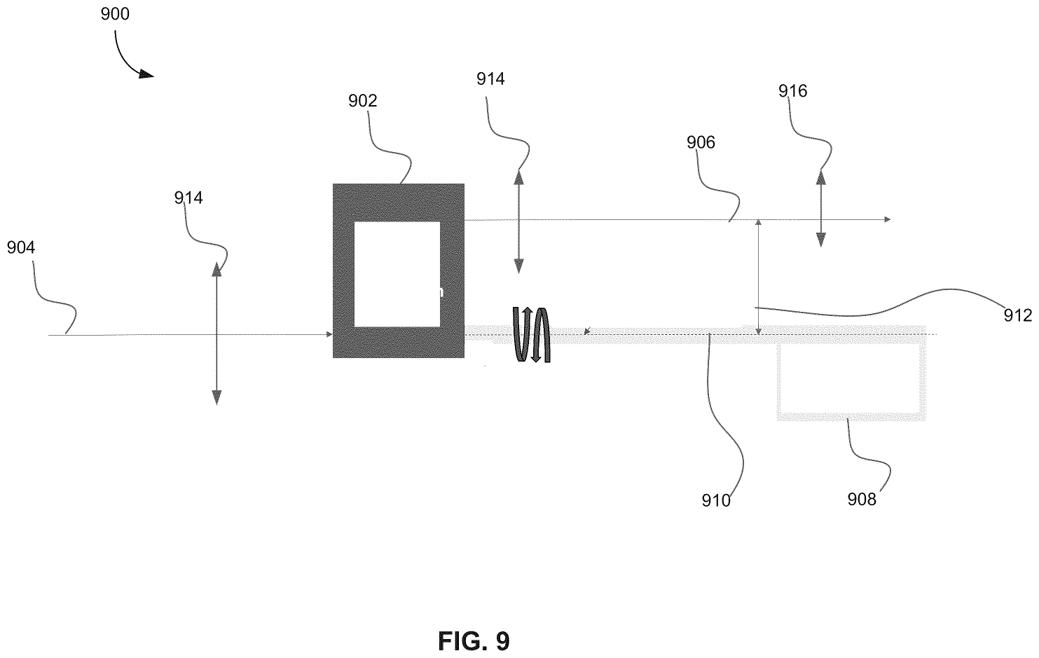

FIGS. 9-14 illustrate embodiments of a treatment probe configuration having an automated assembly for scanning an eye of a patient during treatment. Various descriptions provided above with respect to FIGS. 1-8 are applicable to FIGS. 9-13 unless otherwise indicated. A treatment probe may include any combination of embodiments described in detail above with respect to FIGS. 3A-8. As described herein, a treatment probe may include various configurations to offset a treatment light beam and provide automated scanning capabilities, such as those described in detail above. For example, a treatment fiber may be enclosed in the elongate body of the treatment body, or the treatment fiber may be external to the elongate body of the treatment probe. Furthermore, the treatment probe may include alternative offsetting mechanisms other than the rotating assembly and/or linear stage described in detail above. Various embodiments offset the optical beam from its input centerline (e.g., where the optical input centerline and the rotation centerline are the same) by a controlled amount and the output beam is substantially parallel to the input beam so as to make an arc when rotates, as would be appreciated by one having ordinary skill in the art upon reading the present disclosure.

FIG. 9 illustrates a treatment probe 900 including a beam offset mechanism 902 including an optical beam input path 904 and an optical beam output path 906. The beam offset mechanism 902 may be coupled to a drive mechanism 908 that rotates the beam offset mechanism 902 about an axis of rotation 910. The drive mechanism 908 may include any type of motor including galvos, steppers, or the like. Furthermore, the drive mechanism 908 may include one or more gears in at least some embodiments. The beam offset mechanism 902 may include components that offset 912 the optical beam input path 904 from the optical beam output path 906. As would be appreciated by one having ordinary skill in the art upon reading the description detailed above, the optical beam input path 904 and/or the beam offset mechanism 902 may be translated 914 to adjust 916 an arc radius of the optical beam output path 906.

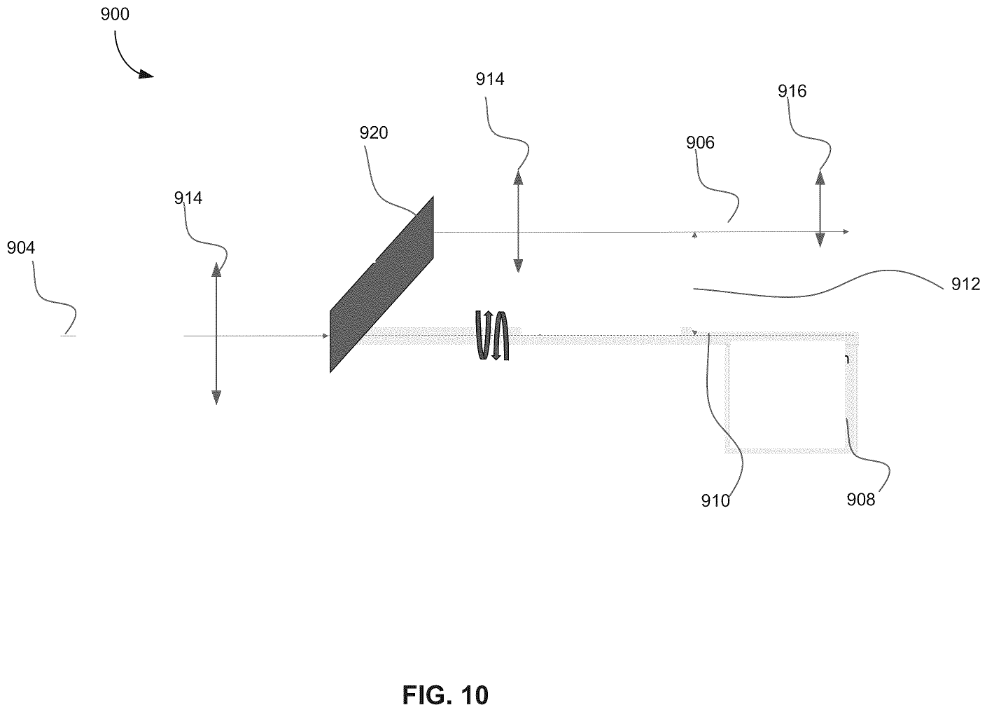

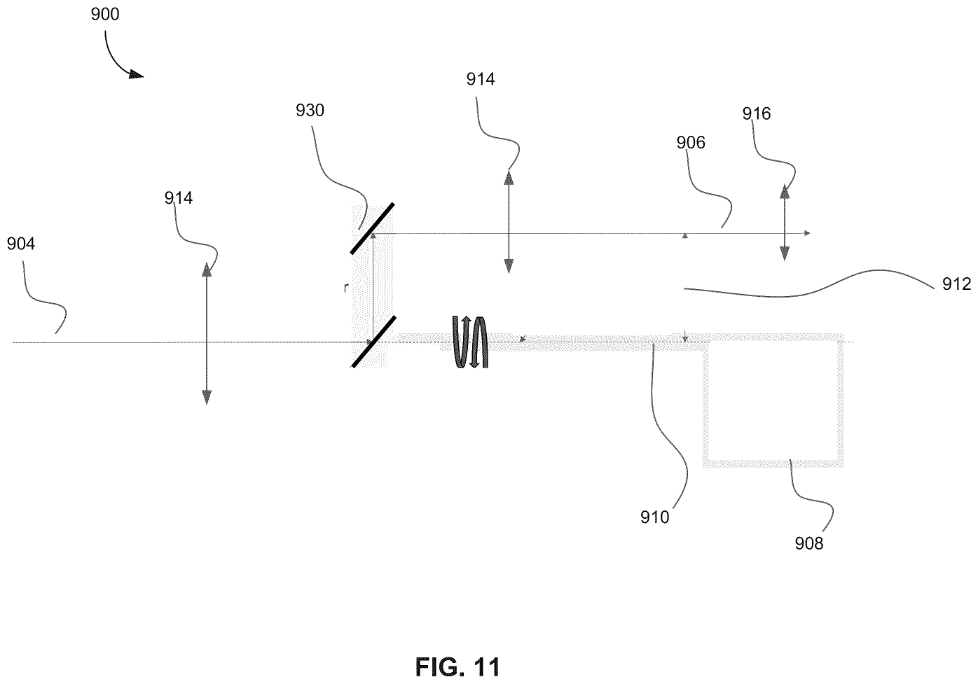

FIG. 10 illustrates an exemplary treatment probe 900 having a rhomboid prism 920 coupled to the drive mechanism 908 to offset 912 the optical beam input path 904 from the optical beam output path 906. FIG. 11 illustrates an exemplary treatment probe 900 having a mirror pair 930 coupled to the drive mechanism 908 to offset 912 the optical beam input path 904 from the optical beam output path 906. As would be appreciated by one having ordinary skill in the art upon reading the description detailed above, the optical beam input path 904 and/or the rhomboid prism 920 or the mirror pair 930 may be translated 914 to adjust 916 an arc radius of the optical beam output path 906. According to at least some embodiments, the optical beam input path 904 may be introduced at an angle (e.g., toward any of the beam offset mechanism 902, the rhomboid prism 920, the mirror pair 930, etc.) that would result in a corresponding translation and angle variation of the optical beam output path 906 and the offset 912.

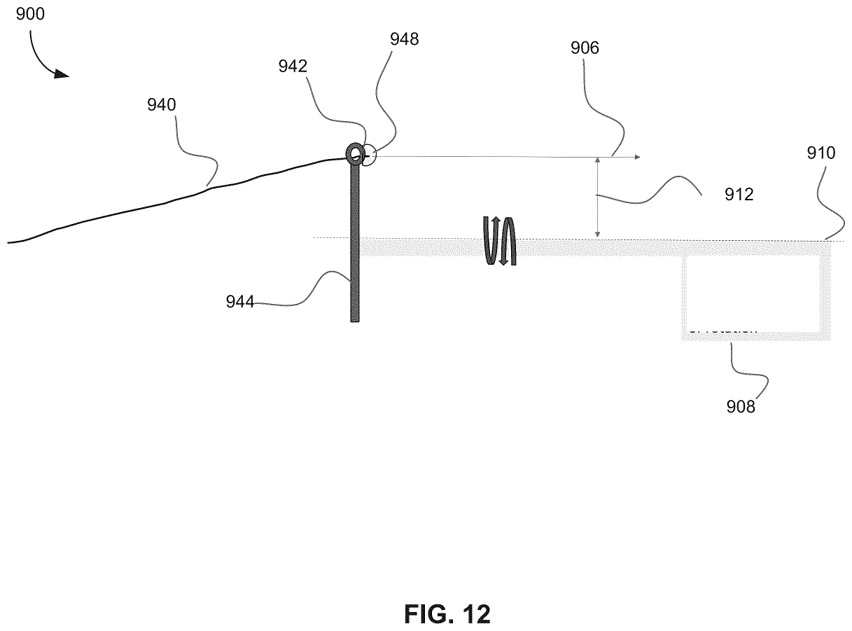

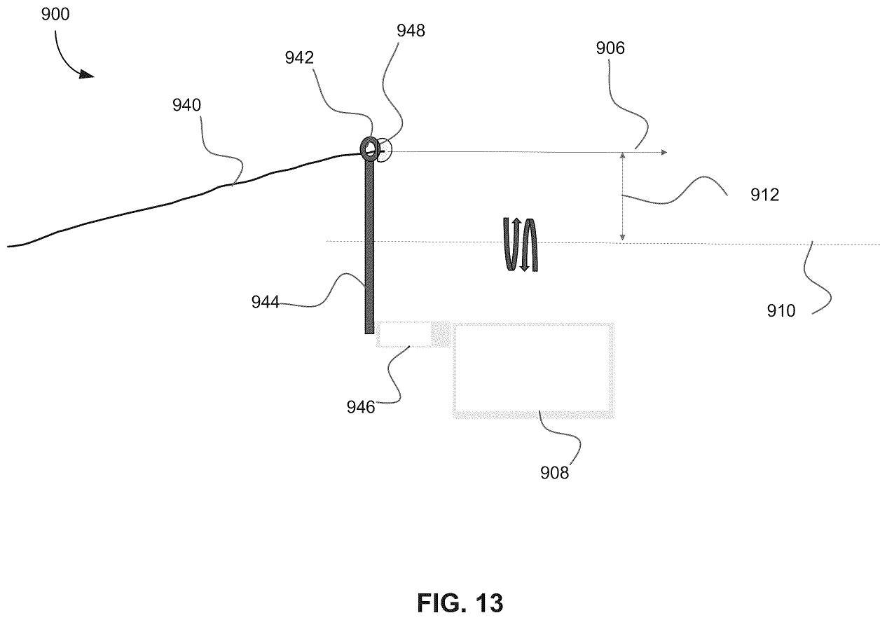

FIGS. 12 and 13 illustrate further exemplary embodiments of a treatment probe 900 having a distal end 942 of a fiber optic 940 coupled to a rotating disc 944 rotated by a drive mechanism 908. The rotation of the rotating disc 944 creates the offset 912 of the optical beam output path 906 from the axis of rotation 910. The shape of the optical beam output path 906 may form a circle or long arc as would be appreciated by one having ordinary skill in the art upon reading the present disclosure. In various embodiments, additional optical element(s) 948 may be included to further tune the optical beam output path 906. According to some embodiments, for short arc lengths, the fiber optic 940 may be aligned with, or only slightly offset from, the desired optical beam output path 906. FIG. 13 illustrates an alternative configuration having the drive mechanism 908 driven by one or more gears 946 and coupled to the rotating disc 944.

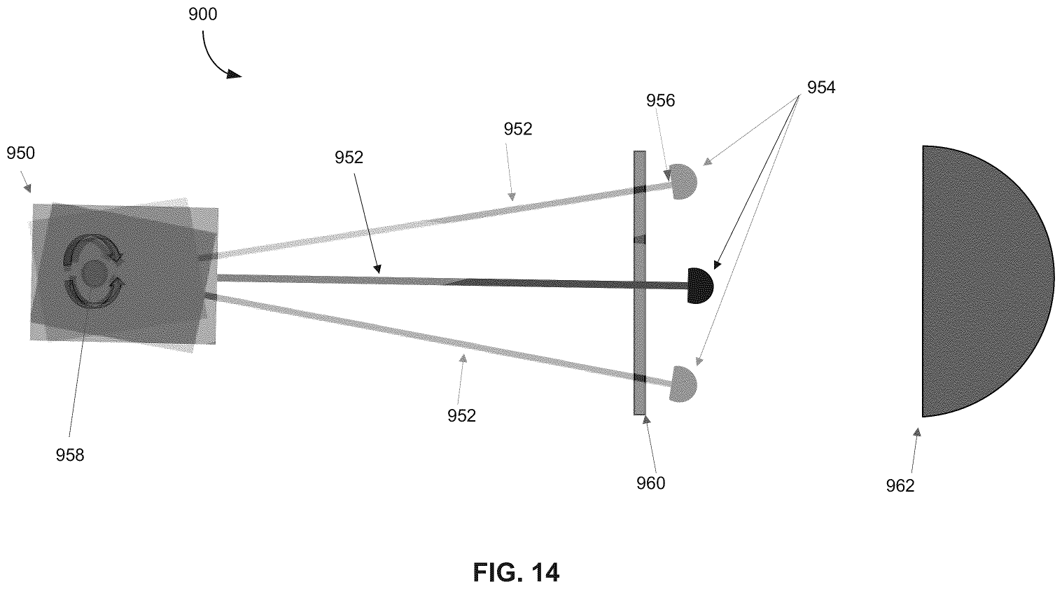

FIG. 14 further illustrates a treatment probe 900 having a laser source 950, such as a laser diode, that is rotated with fiber 952 coupled thereto. For example, the laser source 950 and the fiber 952 are rotated as one with the axis of rotation 958 being perpendicular to the plane of the image and located at a center of the laser source 950. An optical element 954 may be coupled to the distal end 956 of the fiber 952 to focus the light transmitted through the fiber 952 at a target some distance away. An axis of rotation 958 is perpendicular to the fiber 952 such that top view 960 appears linear and side view 962 appears as an arc less than 180 degrees. The fiber 952 may be flexible (not floppy) such that, as the fiber 952 is swept across a structure positioned perpendicularly to the fiber (e.g., side view 962) the fiber 952 flexes to follow the top edge of the structure (e.g., to form an arc shape) and projects an arc shape at the treatment surface. that top view 960 where the axis of rotation 958 is perpendicular to the plane of the image, located near the center of the laser source.

In some embodiments, an automated offset mechanism may include an optical two-dimensional beam steering system implemented with two rotating mirrors positioned orthogonally. The mirrors may be rotated with galvos, stepper motors, or the like, in various combinations. The axis of rotation may be orthogonal or not orthogonal depending on the implementation.

Various embodiments of the present disclosure may be applied to treatment probes for treating many conditions of the eye. For example, at least some embodiments of the treatment probes described herein may be used to treat the back of the eye or to perform other procedures. At least some of the embodiments described herein may be used in treatment probes for treating ocular pathology including for performing retinal, panretinal, focal and pattern photocoagulation of vascular and structural abnormalities of the retina and choroid, or the like.

According to various embodiments described herein, the combination of rotation with linear translation produces a region that is an annulus or an annulus sector. A target portion of the eye for treatment may be the ciliary body and/or immediately adjacent tissue/structures of the ciliary body. In particular, the ciliary body is an annular structure with an inner radius and an outer radius. Within the radius range of the ciliary body different radiuses correspond to different portions of the ciliary body. The inner most portion, roughly the anterior (radially) ⅓, is the pars plicata. The pars plicata region may be treated for IOP control because the pars plicata region holds the aqueous producing tissues. Thermally injuring the aqueous producing tissues reduces IOP by reducing aqueous production. The pars plana is roughly the posterior (radially) ⅔ of the ciliary body. The pars plana region may be treated for IOP control because it has significant muscle fibers that can be contracted by thermal injury to enhance outflow. The central (radially) section of the annular ciliary body is the anterior portion of the pars plana. Inducing thermal injuring to the central ciliary body (e.g., anterior pars plana) enhances the radial contraction of the ciliary body muscle due to the relatively significant muscle mass. The posterior (radially) section of the annular ciliary body is the posterior portion of the pars plana. Inducing thermal injuring to the posterior pars plana enhances the radial contraction of the ciliary body muscle but with less clinical effect due to the reduced muscle mass and thickness.

Accordingly, using various embodiments of the present disclosure, when creating an annulus shaped energy delivery zone by controlling the arc radius, energy may be delivered to different radial regions of the ciliary body to reduce IOP by different mechanisms. Thermally injuring the anterior ⅓ of the ciliary body primarily affects aqueous production. Thermally injuring the mid ⅓ of the ciliary body (anterior pars plana) primarily affects outflow due to the significant volume of ciliary body muscle contraction. Thermally injuring the posterior ⅓ of the ciliary body (posterior pars plana) primarily affects outflow due to the modest volume of ciliary body muscle contraction. The radial accuracy and consistency of energy delivery is critical to achieve safe and efficacious results. Energy delivery by hand to an annulus sector of the ciliary body with high accuracy is difficult. The mechanization of the delivery probe provides consistent and accurate energy delivery to the eye, both in radial accuracy (site closed loop control) and sweep speed (site closed loop control). Different treatment regions may be determined by the size of the ciliary body in the eye of the patient.

According to at least some embodiments, the 810 nm wavelength may be used as this wavelength balances transmission through the sclera (lower scattering and lower absorption) and absorption in the ciliary body. This wavelength achieves relatively high absorption in the ciliary body compared to the sclera such that a therapeutic temperature can be achieved in the ciliary body with minimal risk of thermal injury the sclera and/or adjacent structures.

In various embodiments, a spot size of a treatment probe described herein may be 600 μm. Continuous movement (e.g., sweeping) is more uniform than treating discrete spots. For example, the heating in the ciliary body is more uniform when sweeping continuously in contrast to stopping to deliver energy in one spot for some time then moving to the next spot. However, some applications may be better suited to discrete spot treatment and treatment probes described herein may be used for discrete spot treatment.

At least some embodiments of the present disclosure have a range of parameter sets to achieve a clinically effective n energy delivery range (resulting in time/temp profile in the target tissue). Power settings may be from hundreds of mW to less than 10 W of average power. For discrete spots, the pulse durations may range from hundreds of ms to tens of seconds. For discrete spots, the power and pulse duration combination is optimized to safely achieve the desired clinical effect. For sweeping or near sweeping applications (e.g., such as applications where the operator and/or treatment probe are effectively moving while firing), the sweep speeds can range from a few mm/s (e.g., less than 5 mm/s) to tenths of mm/s (e.g., greater than 0.1 mm/s). For sweeping, the power and sweep speed combination is optimized to safely achieve the desired clinical effect.

While several embodiments and arrangements of various components are described herein, it should be understood that the various components and/or combination of components described in the various embodiments may be modified, rearranged, changed, adjusted, and the like. For example, the arrangement of components in any of the described embodiments may be adjusted or rearranged and/or the various described components may be employed in any of the embodiments in which they are not currently described or employed. As such, it should be realized that the various embodiments are not limited to the specific arrangement and/or component structures described herein.

In addition, it is to be understood that any workable combination of the features and elements disclosed herein is also considered to be disclosed. Additionally, any time a feature is not discussed with regard in an embodiment in this disclosure, a person of skill in the art is hereby put on notice that some embodiments of the invention may implicitly and specifically exclude such features, thereby providing support for negative claim limitations.

Having described several embodiments, it will be recognized by those of skill in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included.

As used herein and in the appended claims, the singular forms “a”, “an”, and “the” include plural referents unless the context clearly dictates otherwise. Thus, for example, reference to “a process” includes a plurality of such processes and reference to “the device” includes reference to one or more devices and equivalents thereof known to those skilled in the art, and so forth.

1. A treatment probe for treating an eye of a patient, the treatment probe comprising:
an elongate body defining a handle and having a proximal end and a distal end;
a treatment fiber configured for delivering treatment light energy to the eye from a distal end of the treatment fiber;
an automated assembly for directing the treatment light energy along a treatment path to the eye from the distal end of the treatment fiber; and
a tip member disposed on the distal end of the elongate body, the tip member including:
one or more contact surfaces for positioning on a surface of the eye, the one or more contact surfaces extending from the distal end of the elongate body.

2. The treatment probe of claim 1, wherein the treatment fiber is housed in the elongate body.

3. The treatment probe of claim 1, wherein the treatment fiber is external to the elongate body.

4. The treatment probe of claim 1, wherein the automated assembly includes a motorized wheel and gear mechanism.

5. The treatment probe of claim 1, wherein the automated assembly includes a motorized wheel mechanism.

6. The treatment probe of claim 1, wherein the treatment path is an arc radius corresponding to a quadrant of the eye.

7. The treatment probe of claim 1, further comprising an aiming beam housed in the elongate body for positioning the treatment probe relative to the eye.

8. The treatment probe of claim 1, wherein the tip member is replaceable at a conclusion of a treatment session.

9. The treatment probe of claim 1, wherein the treatment probe is configured to deliver a plurality of pulses of the treatment light energy to the eye to induce a therapeutic response without coagulating tissue of the eye.

10. The treatment probe of claim 1, wherein the treatment probe is configured to deliver continuous wave (CW) treatment light energy to the eye to induce a therapeutic response of the eye.

11. The treatment probe of claim 1, further comprising a rechargeable battery disposed within the elongate body for powering a treatment fiber energy source coupled to the treatment fiber.

12. The treatment probe of claim 1, further comprising a user display for displaying one or more of display parameter settings, battery information, a mode indication, and an alert indication.

13. A method of treating an eye of a patient, the method comprising:
providing a treatment probe comprising:
an elongate body having a proximal end and a distal end;
a treatment fiber housed in the elongate body; and
an automated assembly for directing treatment light energy along a treatment path to the eye from the distal end of the treatment fiber; and
a tip member disposed on an end of the elongate body, the tip member including:
one or more contact surfaces for positioning on a surface of the eye, the one or more contact surfaces extending from the distal end of the elongate body;
positioning the treatment probe in a first position relative to the eye, wherein the one or more of the contact surfaces of the tip member is positioned on a surface of the eye;
maintaining the treatment probe in the first position relative to the eye; and delivering treatment light energy from a distal end of the treatment fiber to treat tissue of the eye.

14. The method of claim 13, further comprising treating a ciliary process of the eye with the treatment light energy.

15. The method of claim 13, wherein positioning the treatment probe includes confirming a relative position of the treatment probe to the eye using an aiming beam disposed within the elongate body of the treatment probe.

16. The method of claim 13, wherein the delivering the treatment light energy from the distal end of the treatment fiber comprises delivering a plurality of pulses of the light energy so as to induce a therapeutic response without coagulating the tissue of the eye.

17. The method of claim 13, wherein the delivering the treatment light energy from the distal end of the treatment fiber comprises delivering continuous wave (CW) treatment light energy to the eye to induce a therapeutic response of the eye.

18. The method of claim 13, wherein the treatment path is an arc radius corresponding to a quadrant of the eye.

19. The method of claim 13, further comprising:
positioning the treatment probe in a second position relative to the eye, wherein the one or more of the contact surfaces of the tip member is positioned at a different location on the surface of the eye;
maintaining the treatment probe in the second position relative to the eye; and
delivering treatment light energy from the distal end of the treatment fiber to treat tissue of the eye.

20.-71. (canceled)

72. A treatment probe for treating an eye of a patient, the treatment probe comprising:
an elongate body defining a handle and having a proximal end and a distal end;
a treatment fiber housed in the elongate body and configured for delivering treatment light energy to the eye from a distal end of the treatment fiber;
a rotating assembly for directing the treatment light energy along a treatment path to the eye from the distal end of the treatment fiber and through the rotating assembly; and
a tip member disposed on the distal end of the elongate body, the tip member including:
one or more contact surfaces for positioning on a surface of the eye, the one or more contact surfaces extending from the distal end of the elongate body.
